(19) United States
(12) Patent Application Publication
(10) Pub. No.: US 2008/0097524 A1
(43) Pub. Date: Apr. 24, 2008

(54) Surgical Thread and Surgical Device

(75) Inventors: Harry Goransson, Espoo (FI); Anna Viinikainen, Helsinki (FI); Pentti Rokkanen, Helsinki (FI); Katja Huovinen, Tampere (FI); Minna Kellomaki, Kangasala (FI); Pertti Tormala, Tampere (FI)

Correspondence Address: VENABLE LLP, P.O. BOX 34385, WASHINGTON, DC, 20043-9998, US

(73) Assignee: Bioretec Oy, Tampere (FI)

(21) Appl. No.: 11/661,373

(22) PCT Filed: Aug. 31, 2005
(86) PCT No.: PCT/FI05/50308

§ 371 Date: Dec. 17, 2007

(30) Foreign Application Priority Data
Aug. 31, 2004 (FI) 20045316

(51) Int. Cl. A61B 17/06; A61B 17/04

(52) U.S. Cl. 606/222; 606/228

(57) ABSTRACT

A surgical thread and a surgical device. The surgical thread includes at least two adjacent elongated elements that are connected to each other and include filament fibers such that a maximum cross-sectional diameter of the thread is substantially greater than the cross-sectional diameter of the thread in the direction perpendicular to the maximum diameter, and the elements are connected by a structure that is arranged to be broken at least at the ends of the elements such that the thread can be divided into single elements. The surgical device includes a needle and a surgical thread attached to the surgical needle.

The present invention relates to a surgical thread comprising filament fibers, and a surgical device comprising a surgical needle and a surgical thread comprising filament fibers and being attached to the surgical needle.

The surgical thread according to the invention is used particularly for fixing the tendons of fingers. Prior art in the fixing of tendons in fingers is represented by U.S. Pat. No. 4,971,075. This publication discloses a surgical thread which is a multifilament thread that does not degrade in tissue conditions. The surgical thread is attached to a surgical needle.

In the use of surgical threads of the above-mentioned publication, problems have been caused by the following factors:
The thread bites into the tendon so that it causes damage to the circulation in the tendon, and
the thread does not hold during movements of the tendon, wherein, for example, splinting must be used to prevent the movements. This, in turn, results in slower healing, because moving the tendon has been found to accelerate the healing and to prevent the formation of harmful adhesions.

The surgical thread and the surgical device according to the invention are characterized in that they comprise at least two adjacent elongated elements which are connected to each other and comprise filament fibers in such a way that the maximum cross-sectional diameter of the thread is substantially greater than the cross-sectional diameter of the thread in the direction perpendicular to the maximum diameter, and the elements are connected by a structure that is arranged to be broken at least at the ends of the elements in such a way that the thread can be divided into single elements.

The cross-sectional diameter of the thread according to the invention has a flat shape, wherein the tension caused by it is divided more evenly in the tendon than in the case of a thread with a relatively circular cross-section, due to the larger surface area of the flat thread. In connection with the flat thread, one knot becomes so large that it will hinder the movability of the tendon. When the thread consists of several elements which can be separated from each other, the single elements can be tied separately from each other to form several small knots, wherein the movability of the fixed tendon is better and the tendon heals faster than in the case of one large knot.

The surgical thread according to the invention is used for fixing tendons, particularly for fixing tendons in fingers. The fixing of other tendons is also feasible; for example, it is possible to fix the calcaneal tendon with the surgical thread according to the invention. In principle, the fixing is performed in such a way that the parts of the tendon are joined to each other with a loop of the thread whose ends are tied with a knot. For fixing a tendon, several thread loops are often used, but a fixing technique utilizing a single thread loop is also known: modified Kessler. When using the surgical thread according to the invention, it should be noted that the fixing of the tendon can be performed by using only one thread loop, because the strength of the surgical thread according to the invention is greater than that of known threads used for a corresponding purpose.

The maximum cross-sectional diameter of the surgical thread according to the invention is substantially greater than the cross-sectional diameter of the thread in a direction perpendicular to the maximum diameter, wherein the thread becomes flat. The maximum cross-sectional diameter of the thread depends on the number of adjacent elements and the space required by the structure connecting the elements. Normally, the cross-sectional diameter of the thread in the direction perpendicular to the maximum diameter is substantially the same as the diameter of the element. Consequently, if the number of elements is at least two, the cross-sectional diameter is substantially circular and the diameter of the element is d, then the maximum diameter of the surgical thread is at least 2d, even though the elements were substantially attached to each other.

In its simplest form, the elongated element is a monofilament fiber, but preferably it consists of more than one filament fibers. Within the element, the filament fibers may be arranged as partial elements twisted around each other. The filaments of the partial elements may constitute a fiber bundle of straight fibers, or the fiber bundle may be twisted. The element can also form a braided, knitted or woven band or a tube which may encircle a fiber bundle consisting of fibers. The elements comprise material that is degradable in tissue conditions; preferably, the elements are made completely of a material that is degradable in tissue conditions.

The structure of the element may differ from that described above, but it is obvious that it must be elongated and that it must have a structure causing as little damage as possible to the tissue when passing through it. However, the above-described structure of the element has clear advantages: it has a small cross-section and the elongation is well under control.

The surgical thread comprises at least two elongated elements connected to each other, and the elements are joined by a structure that can be broken to separate the elements from each other; in other words, the thread can be split into parts. The connecting structure normally comprises a connecting thread or threads that have been twisted and threaded around the elements or that may also penetrate the elements to form a flat thread structure. Alternatively, the connecting thread can be threaded between the partial elements in the cross direction of the elements, wherein the band-like structure can be formed by one connecting thread zigzagging forward between the elements. The connecting thread is normally thinner than the elements. It is also possible that the connecting structure formed between the elements is a film that can be broken in the longitudinal direction of the elements. It is obvious that the connecting film is substantially weaker than the elements. The structure connecting the elements comprises a material that is degradable in tissue conditions; preferably, the structure connecting the elements is made completely of a material degradable in tissue conditions.

Preferably, the material chosen as the material for the elements and the connecting structure is a material that is degradable under tissue conditions, for example, poly-α-hydroxy acid, because it is the more advantageous for the fixing of the tendon, the less foreign matter is introduced and left by the fixing in the tendon. A material that is advantageous for both the elements and the structure connecting the elements and is degradable under tissue conditions, is, for example, a polylactide copolymer, because the drop in its strength as a function of time matches the time required for healing of the tendon. By varying the relative amounts of the starting dimers (LL; DD; DL dimers) of the polylactide copolymer, the rate of change in the strength of the polymer can be adjusted as desired. In other words, certain poly-D-L-lactides degrade at a suitable low rate so that there is enough time for the tissue structure of the tendon to regenerate and to replace the thread, wherein the total strength of the tendon fixing remains relatively constant. It has been found in some tests that a given poly-D-L-lactide retains half of its initial strength even after 10 weeks (in vivo). In addition to the poly-D-L-lactide, applicable materials include, for example, polyglycolide (PGA) and polydioxanone (PDS) as well as the copolymers of their initial monomers together with the above-mentioned lactide dimers. Also other synthetic biodegradable polymers are suitable as raw materials for the surgical thread according to the invention. Such polymers are listed, for example, in U.S. Pat. No. 6,692,497.

The threads according to the invention may also include various additives and/or bioactive additives, such as drugs. Such additives are also listed in U.S. Pat. No. 6,692,497.

An advantageous embodiment of the surgical thread according to the invention is a thread consisting of three elements and two connecting threads. The elements consist of two partial elements twisted around each other. The partial elements consist of several filament fibers. The diameter of the filament fibers is normally not more than 0.1 mm, preferably smaller than 0.1 mm. The connecting thread is substantially thinner than the elements. The connecting thread is preferably formed of two filaments twisted around each other. The connecting threads have been wound around the elements in such a way that the first connecting thread has been wound around the first and the second element in an alternating manner, and the second connecting thread has been wound around the second and the third element. The directions of winding of the connecting threads are opposite to each other, and they intersect at the second element. The smaller diameter of the finished surgical thread is normally smaller than 1 mm, preferably smaller than 0.7 mm, and the larger diameter is normally smaller than 2 mm, preferably smaller than 1.7 mm.

The surgical thread according to the invention is normally attached to at least one surgical needle by which the suturing operations required for fixing the tendon can be performed. The surgical thread together with the surgical needle, to which it is attached, constitute a surgical device. Instead of one needle, the surgical device may comprise two needles, one needle at each end of the surgical thread.

After a thread loop required for fixing the tendon has been placed to connect different parts of the damaged tendon, part of the connecting structure, such as the connecting threads, is unraveled at the ends of the elements in such a way that the surgical thread is split into single elements, wherein they can be tied, one by one, two elements together, so that there will be more knots for one surgical thread but these are smaller than a joint formed by one knot only. Normally, cutting with scissors is used for unraveling the thread into single elements. The surface of the elements may be subjected to a treatment to increase the friction between the elements, which improves the holding of the knots. The treatment to increase the friction may be, for example, a chemical treatment or a plasma treatment. Normally, the knot type used is the so-called reef knot.

For fixing the flexor tendon of a finger, a force of 25 N simulates active mobilization without a resistance, 35 N covers the upper values of the forces of active mobilization of FDP and FPL tendons without a resistance, 45 N simulates mobilization with a light resistance (FDP=Flexor digitorum profundus, deep flexor tendons of fingers, FPL=Flexor pollicis longus, long flexor tendon of the thumb). For the knots used for the fixing to have some kind of a safety margin, the maximum fixing force (the strength of the tendon joint) should be about 80 N.

In the following, the invention will be described by means of an example and with reference to the appended drawings, in which

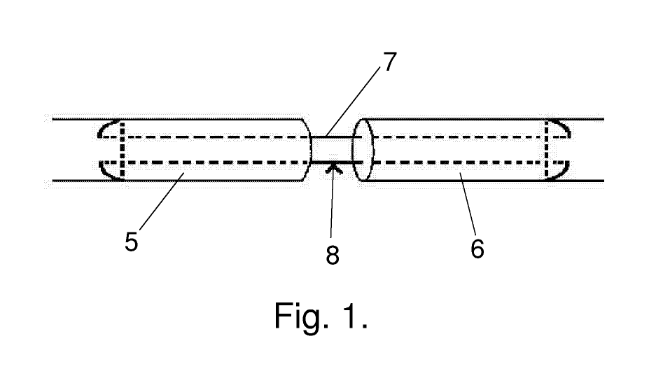

FIG. 1 illustrates a known technique for fixing a tendon, modified Kessler,

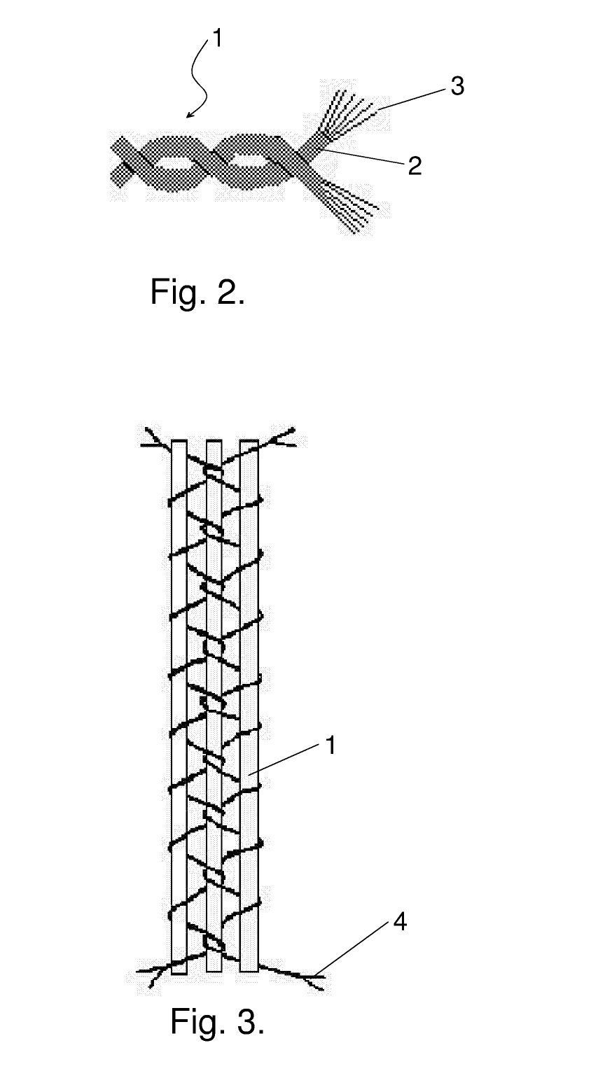

FIG. 2 shows a single element belonging to a surgical thread, and

FIG. 3 shows a surgical thread according to the invention.

FIG. 1 illustrates a known technique for fixing a tendon, modified Kessler. According to this technique, the tendon can be fixed by using only one thread loop formed of a surgical thread 7 to connect the parts 5 and 6 of the tendon. For connecting the ends of the thread loop, an ordinary knot 8 is presented. The span of the surgical thread 7 inside the tendon is shown with a broken line.

FIG. 2 shows a single element 1 of the surgical thread according to the invention. In the case of FIG. 2, the element 1 comprises two partial elements 2 twisted around each other. The partial elements 2 have been made of several filament fibers 3. The filament fibers 3 are degradable under tissue conditions, preferably poly-D-L-lactide fibers.

FIG. 3 shows a surgical thread according to the invention, comprising parallel elements 1. In the case of FIG. 3, the number of parallel elements is three. The elements 1 may be of the type shown in FIG. 2, although this is not necessary. The structure connecting the elements 1 comprises, in the case of three parallel elements 1, two connecting threads 4, each having been wound around two elements in an alternating manner so that both connecting threads have been wound around the middle element 1 and the connecting threads 4 intersect at the middle element 1, and the connecting thread 4 proceeds all the time with a given pitch in the longitudinal direction of the elements 1. The directions of rotation of the connecting threads 4 are opposite to each other. The single connecting thread 4 has been wound around two elements in such a way that if the connecting thread 4 has been wound clockwise around the first element 1, it has been wound counterclockwise around the second element 1. Naturally, the directions of rotation may also be the other way around, but it is essential that the direction of rotation always changes when proceeding to the next element. When the connecting threads 4 are viewed in a direction perpendicular to the longitudinal direction of the elements 1, the connecting threads form a pattern with the shape of the number 8.

After a tendon has been fixed, for example, by the technique shown in FIG. 1 by forming a thread loop between the damaged parts of the tendon, a suitable length of the connecting threads 4 can be unwound around the elements 1. Thus, the ends of the elements can be tied one by one, wherein three small knots are formed instead of one large knot, which make better mobility and healing of the tendon possible.

### EXAMPLE

The values presented in this example are values obtained from sterilized material (25 kGy being the minimum dose of gamma radiation used for sterilization). Because it is difficult to measure the cross-sectional area of a tendon fixing in a reliable way, making it more difficult to define the strength, maximum forces have been used as the variables, instead of strength values.

The filament fibers were made by melt spinning of poly-L/D-lactide (PURASORB®, manufactured by PURAC Biochem B.V., Holland; the inherent viscosity being 4.98 dL/g and the ratio of D/L isomers=96/4). The diameter of the obtained filaments with a circular cross-section was 0.08 to 0.09 mm. The maximum loading capacity of a single filament was about 2 N (drawing speed 30 mm/min, 50 mm drawing span).

An element was made of 12 filaments, to have a maximum loading capacity of about 20 N (drawing speed 20 mm/min, 50 mm drawing span) and a diameter of about 0.5 mm.

A flat surgical thread was made of three elements by joining the elements with two connecting threads, each consisting of two filaments (see FIG. 3). The connecting threads were made of the same material as the elements. The maximum loading capacity of the finished surgical thread under drawing was about 60 N (drawing speed 20 mm/min, 50 mm drawing span), and the smaller diameter was smaller than 0.7 mm and the larger diameter was smaller than 1.7 mm.

The performance of the above-mentioned surgical threads was tested with a tendon model. The tendons used in the testing were extensor tendons of rear cloven hooves of frozen slaughter swine, their size corresponding to the size of the flexor tendon of a human finger. When the tendon was fixed with the surgical thread according to the invention, the average maximum loading capacity of the fixing was about 80 N (drawing speed 20 mm/min, drawing span 35 mm).

For comparison, corresponding tendons were fixed with commonly used suture techniques by using a commercial suture thread. Under drawing, the following results were obtained for the maximum loading capacity:

### Six-filament Savage 4-0 with Ticron 76 N,

four-filament Savage 3-0 with Ticron 68 N,
four-filament Savage 4-0 with Ticron 56 N,
modified duplex Kessler 3-0 with Ticron 68 N, and
modified Kessler 3-0 with Ticron 35 N.

In a dynamic endurance test, the tendon fixing made by a flat surgical thread according to the invention endured an average of 4000 cycles of a 35 N load, followed by 4000 cycles of a 45 N load, before the fixing seam was opened by one millimetre (extensor tendon of rear cloven hooves of slaughter swine, frequency lower than 1 Hz, drawing span 35 mm). The opening of one millimetre is considered the critical gap encumbering the healing of a tendon.

The above-described facts do not restrict the invention, but the invention may vary within the scope of the claims.

1-10. (canceled)

11. A surgical thread, comprising:
at least two adjacent elongated elements which are connected to each other and comprise filament fibers in such a way that a maximum cross-sectional diameter of the thread is substantially greater than a cross-sectional diameter of the thread in the direction perpendicular to the maximum cross-sectional diameter; and
a connecting structure connecting the elements, wherein the connecting structure is arranged to be broken at least at ends of the elements such that the thread can be divided into single elements.

12. The thread according to claim 11, wherein the elements comprise more than one filament fiber.

13. The thread according to claim 12, wherein the elements have been divided into partial elements comprising filament fibers, the partial elements being twisted around each other.

14. The thread according to claim 10, wherein the connecting structure comprises connecting threads arranged to tie the elements together.

15. The thread according to claim 10, wherein the connecting structure comprises a film connecting the elements.

16. The thread according to claim 10, wherein the thread comprises three adjacent elements connected by a structure comprising two connecting threads, of which a first connecting thread is wound around a first and a second of the elements in an alternating manner, and a second connecting thread is wound around the second and a third of the elements in an alternating manner in such a way that the first and the second connecting thread intersect at the second element.

17. The thread according to claim 10, wherein the thread comprises a material that is degradable in tissue conditions.

18. The thread according to claim 17, wherein the thread comprises a polymer, copolymer or polymer mixture that is degradable in tissue conditions.

19. The thread according to claim 18, wherein the material degradable in tissue conditions is a poly-α-hydroxy acid.

20. The thread according to claim 19, wherein the material degradable in tissue conditions is poly-D-L-lactide.

21. A surgical device, comprising:
a surgical needle; and
a surgical thread attached to the surgical needle, the surgical thread comprising at least two adjacent elongated elements that are connected to each other and comprise filament fibers such that a maximum cross-sectional diameter of the thread is substantially greater than a cross-sectional diameter of the thread in the direction perpendicular to the maximum cross-sectional diameter, and the elements are connected by a connecting structure that is arranged to be broken at least at the ends of the elements in such a way that the thread can be divided into single elements.
